(19) United States
(12) Patent Application Publication
(10) Pub. No.: US 2007/0299406 A1
(43) Pub. Date: Dec. 27, 2007

(54) DOSING MECHANISM FOR AN INJECTION DEVICE

(76) Inventors: Stefan Burren, Bremgarten (CH); Ulrich Moser, Heimiswil (CH); Roland Schoch, Nennigkofen (CH); Christian Schrul, Burgdorf (CH); Philippe Zurflueh, Pratteln (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/774,844

(22) Filed: Jul. 9, 2007

Related U.S. Application Data
(63) Continuation of application No. PCT/CH05/00740, filed on Dec. 12, 2005.

(30) Foreign Application Priority Data
Jan. 10, 2005 (DE) DE10 2005001159.4

(51) Int. Cl. A61M 5/00

(52) U.S. Cl. 604/207

(57) ABSTRACT

A dose setting device for an injection device including a housing, a forward-feed element and a setting element, the dose setting device including a rotating sleeve connected to the setting element and having an external thread and an internal thread with a selected different pitch from the external thread, wherein one thread of the rotating sleeve engages with the injection device and the other thread engages with the forward-feed element whereby a dose setting movement of the setting element is different from a dose setting movement of the forward-feed element.

### CROSS-REFERENCE TO RELATED APPLICATIONS

This application is a continuation of International Application No. PCT/CH2005/000740, filed on Dec. 12, 2005, which claims priority to German Application No. DE 10 2005 001 159.4 filed on Jan. 10, 2005, the contents of both of which are incorporated in their entirety herein by reference.

### BACKGROUND

The present invention relates to devices for dispensing, injecting, infusing, administering or delivering substances, and to methods of making and using such devices. More particularly, the present invention relates to a dose setting, adjusting or selecting device for setting, adjusting or selecting a dose to be dispensed from an injection device, e.g. an injection pen. More particularly, it relates to a dose setting device for setting one or more predefined fixed dose units or for preparing an injection device in readiness for dispensing one or more preset fixed dose quantities, e.g. from an ampoule inserted in the injection device.

WO 97/36626 discloses an injection device having a housing with a reservoir for a product. A plunger is accommodated in the reservoir, which forces the product out of the reservoir through an outlet of the reservoir when pushed in a forward-feed direction. A toothed rack serves as the plunger rod and pushes the plunger in the forward-feed direction. Also accommodated in the housing is a drive element which can be displaced relative to the housing in and opposite the forward-feed direction, which drives the toothed rack with it when pushed in the forward-feed direction. To this end, the drive element engages with drivers in rows of teeth of the toothed rack. To set the product quantity that will be administered with a stroke, i.e. by operating a dose setting mechanism, the drive element is manually pulled back from a forward position back in the direction opposite the forward-feed direction by a set dose distance length. As this happens, the drivers of the drive element slide over the teeth of the rows of teeth of the toothed rack, flexing elastically as they do so. The toothed rack is prevented from being vented from being pulled back due to locking means which are mounted so as to pre-vent any movement relative to the housing. The locking means co-operate with one of the rows of teeth of the toothed rack to prevent a movement of the toothed rack in the direction opposite the forward-feed direction. They flex elastically to allow the toothed rack to move in the forward feed direction. When the drive knob is operated, the drive element moves across the dose path length set by the toothed rack and plunger so that the set dose is dispensed through the outlet of the reservoir.

### SUMMARY

An object of the present invention is to provide a dose setting mechanism for an injection device which makes it easier to set a dose, and to set and dispense a small dose quantity exactly.

In one embodiment, the present invention comprises a dose setting device for an injection device comprising a housing, a forward-feed element and a setting element, the dose setting device comprising a rotating sleeve connected to the setting element and having an external thread and an internal thread with a selected different pitch from the external thread, wherein one thread of the rotating sleeve engages with the injection device and the other thread engages with the forward-feed element whereby a dose setting movement of the setting element is different from a dose setting movement of the forward-feed element.

In one embodiment, the present invention comprises an injection device comprising a dose setting device comprising a setting element and a rotating sleeve connected to the setting element and having an external thread and an internal thread with selected a different pitch from the external thread, wherein one thread of the rotating sleeve engages with the injection device and the other thread engages with a forward-feed element whereby a setting movement of the setting element results in a different dose setting movement of the forward-feed element.

In one embodiment, the present invention comprises a dose setting device or arrangement for an injection device with a setting element, a rotating sleeve which is connected to the setting element and has an external thread and an internal thread with a different pitch from the external thread, and one thread of the rotating sleeve engages with a part of the injection device, in particular a housing, and the other thread engages with a forward-feed element so that a setting movement of the setting element which results in a dose setting movement of the forward-feed element due to the two thread engagements can be increased or reduced.

A dose setting device for an injection device in accordance with the present invention comprises a setting element, a cylindrical rotating sleeve or a knob, for example, which can be fixedly or rotatably connected to a cylindrical rotating sleeve. The rotating sleeve has an internal thread and an external thread, in some preferred embodiments with the same direction of rotation, which are disposed coaxially with one another so that the rotating sleeve has an internal thread and an external thread in at least one region. In some preferred embodiments, the threaded regions overlap and may be disposed more or less across the entire length of the rotating sleeve on the internal face and the external face of the cylindrical rotating sleeve body. In some preferred embodiments, the threads are provided in the form of kinetic threads so that the threads are not retained by friction, and thread pitches or elements or cams can engage in thread pitches or alternatively circumferentially extending spirals can engage in the thread of respectively oppositely lying counter-threads or can co-operate with them. Provided inside and outside the rotating sleeve are elements of the injection device, which are fitted so that they can not rotate relative to one another or which are fitted so that they can not rotate relative to a housing of the injection device, for example. An element inside or outside the rotating sleeve and contacting or lying on it may be a part of the housing of the injection device, for example. The elements lying inside and outside the rotating sleeve may themselves be cylindrical, for example, or may be formed along only one or more part-regions in the circumferential direction of the rotating sleeve. They may comprise counter-threads, for example in the form of individual cams or circumferentially extending spiral-shaped grooves or webs which engage in the internal and external thread of the rotating sleeve or co-operate with corresponding thread elements of the rotating sleeve. In some embodiments of the present invention, the internal thread of the rotating sleeve has a different pitch from the external thread of the rotating sleeve.

When a forward-feed element of the injection device is fitted inside the rotating sleeve, for example disposed in threaded engagement with the rotating sleeve, the forward-feed element can be moved in a defined manner by a predefined distance in the proximal and distal direction of the injection device by turning the rotating sleeve, which is in threaded engagement with a housing part of the injection device lying against the rotating sleeve, for example on the external face of the rotating sleeve. If the internal thread of the rotating sleeve has a smaller pitch than the external thread of the rotating sleeve, when the rotating sleeve is screwed or turned by the axial length D out of the housing of the injection device with which the rotating sleeve is in the thread engagement, the forward-feed element is moved in the same direction by a shorter distance d. Results include a reduction of a setting movement and compact design. A short functional path d can be increased to a longer path D to simplify the setting, e.g., for a fixed dose.

In some embodiments, if a driver such as a suitable catch element is connected to the forward-feed element, e.g., provided on a flexible element or arm of the forward-feed element. The catch element, e.g., a cam or lug projecting radially inwards from the flexible element, can be guided across the teeth of a toothed rack serving as a plunger rod or the thread of a threaded rod. Depending on the extent of the axial movement of the forward-feed element relative to the toothed rack, the elastically mounted cam or lug is moved across one or more teeth of the toothed rack, thereby making one or more “clicking” noises and fixing the dose to be dispensed from the injection device. Set in this manner, the dose is dispensed due to the movement of the forward-feed element or the forward-feed sleeve in the distal direction caused by pulling back or rotating back the rotating sleeve, which is transmitted to the toothed rack due to an engagement of the cams or lugs and then on to a stopper lying directly or indirectly adjoining the toothed rack, which is pushed into an ampoule to force out a substance contained in it.

As regards the operating principle of a dose setting device which has a forward-feed sleeve or a forward-feed element, reference may be made to the German patent application bearing application number 10 2004 041 151.4, owned by the owner of the present application, the disclosures of which relating to the design of a dose setting device and the co-operation of flexible elements carrying cams or lugs and mounted on a forward-feed sleeve and an outer sleeve or a housing to co-operate with a toothed rack, are incorporated in this application.

In some preferred embodiments of the present invention, as regards the threads of differing pitch provided on the internal and external face of the rotating sleeve, the pitch of the thread which engages with the forward-feed element may be smaller than the pitch of the oppositely lying thread, which engages with the housing or with another component of the injection device, for example, with respect to which the forward-feed element is mounted so that it can not rotate, so that a forced rotation of the rotating sleeve relative to the housing caused by a threaded engagement of the rotating sleeve during a setting operation is not converted into a rotation of the forward-feed element, which is also in a threaded engagement with the rotating sleeve, but into an axial movement of the forward-feed element relative to the housing. This being the case, the forward-feed element may be provided with an external or internal thread both inside and outside the rotating sleeve. Likewise, it is possible for the pitch of the thread with which the rotating sleeve and forward-feed element engage to be bigger than the pitch of the thread provided on the other side of the rotating sleeve, in which case a short setting movement is converted into an axial movement of the forward-feed element of a bigger ratio.

In some embodiments, a setting element, for example a control knob or the like, is provided on or fixedly connected to the rotating sleeve so that the rotating sleeve can be screwed or turned out of the injection device by turning the control knob. It is also possible for the rotating sleeve to be rotatably connected to a setting element or control knob so that the control knob can be pulled out of the housing of the injection device without a rotating movement, for example, and the rotating sleeve, which is rotatably mounted in the control knob, can be moved, turned or screwed out of the housing of the injection device.

In some preferred embodiments of the present invention, the threads provided on the rotating sleeve are not retained by friction, in which case the pitch angle of the thread can be selected so that the tangent of this pitch angle is greater than the coefficient of friction at points where materials lie against one another and establish a thread engagement. It would also be possible to use lubricant, such as Teflon or the like, so that the thread engagements are not retained by friction. A spring, for example a torsion spring, may be provided and may be tautened or tensioned as the rotating sleeve is pulled out or screwed out. The spring may be connected to the rotating sleeve so that when the rotating sleeve is being pushed in or screwed in, the spring force acts in the direction of rotation, in which case threads retained by friction may also be used and the friction of the thread overcome by the spring.

In some preferred embodiments of the present invention, the external and internal threads of the rotating sleeve are disposed coaxially with one another, i.e. the threads overlap in the axial direction, thereby resulting in a way of achieving a reduction ratio and increasing ratio to permit a design which is compact and reduces the length of an injection device.

In some preferred embodiments, of the present invention, a radial and/or axial stop or stops are provided which are able to restrict the movement of the forward-feed element and/or the rotating sleeve in the radial and/or axial direction relative to the housing of the injection device. For example, two stops may be provided on the forward-feed element spaced apart from one another in the axial direction, which cooperate with a stop element of the housing so that the forward-feed element can be moved in the axial direction of the injection device relative to the housing by only a predefined distance d, in which case a dose quantity which can be dispensed from the injection device can be preset so that it corresponds to the distance d. In some embodiments, radial stops may be provided on the rotating sleeve and/or on the elements cooperating with the rotating sleeve, in other words the forward-feed element and another element of the injection device, such as the housing, which restrict a rotating movement of the rotating sleeve and permit a maximum rotation of only 180° or two full rotations, for example. A fixed dose can be preset by such stops so that a user pulls the setting element out as far as a stop of an element, for example, and the preset dose is dispensed when the setting element is pushed in.

In some preferred embodiments, a marking is provided on the rotating sleeve and/or on a setting element, such as a control knob, connected to the rotating sleeve. The marking can be read or seen by a user through an orifice or transparent material in the state when it is not pushed into the injection device, the rotating sleeve and/or a control element connected to the rotating sleeve having been pulled out of the injection device or housing of the injection, and the extraction distance thus enables a user to take a reading of the set fixed dose, which is then discharged from the injection device when the rotating sleeve or control knob is pushed back due to the retraction movement transmitted to the toothed rack and the stopper.

The present invention encompasses an injection device, containing a substance to be dispensed or in which an ampoule can be inserted, which has a dose setting device of the type described above which can be operably coupled with a forcing body or stopper of the injection device so that a dose to be dispensed can be set and dispensed by the rotating sleeve.

### BRIEF DESCRIPTION OF THE DRAWINGS

FIGS. 1A-1C illustrate an embodiment of a setting device in accordance with the present invention, including during setting and after dispensing a dose;

FIGS. 2A-2B show a cross-section of the dose setting device in accordance with the present invention; and

FIG. 3 shows an embodiment of an injection device in accordance with the present invention with a dose setting device in accordance with the present invention.

### DETAILED DESCRIPTION

With regard to fastening, mounting, attaching or connecting components of the present invention, unless specifically described as otherwise, conventional mechanical fasteners and methods may be used. Other appropriate fastening or attachment methods include adhesives, welding and soldering, the latter particularly with regard to the electrical system of the invention, if any. In embodiments with electrical features or components, suitable electrical components and circuitry, wires, wireless components, chips, boards, microprocessors, inputs, outputs, displays, control components, etc. may be used. Generally, unless otherwise indicated, the materials for making the invention and/or its components may be selected from appropriate materials such as metal, metallic alloys, ceramics, plastics, etc.

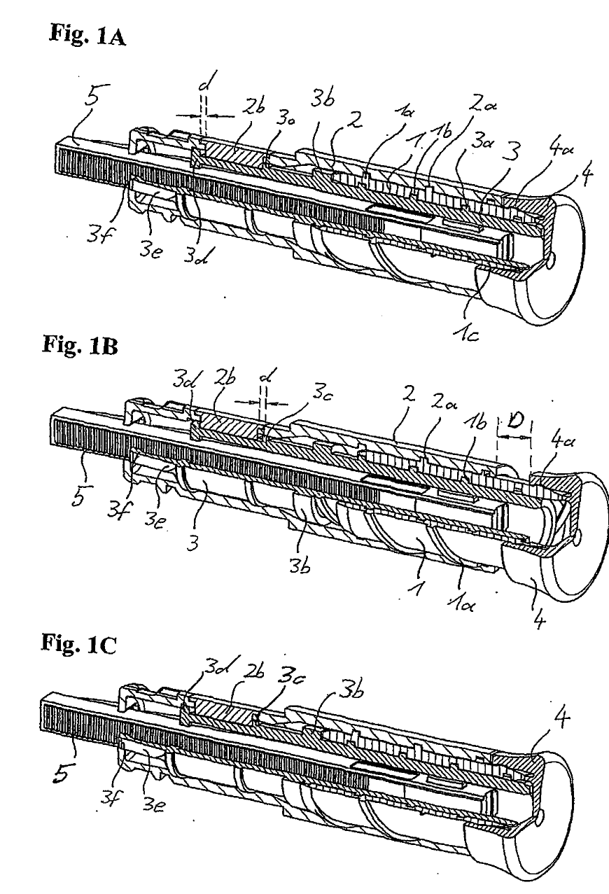

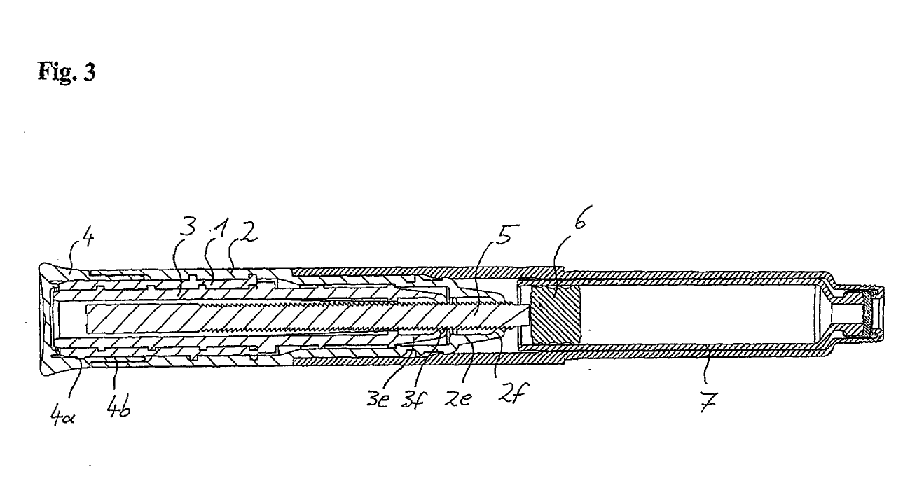

FIGS. 1A to 1C illustrate a dose setting device in accordance with the present invention for an injection device illustrated by way of example in FIG. 3, with a housing 2 which has an internal thread 2a with a first pitch of 49°, for example. Disposed coaxially in the housing and mounted so as to be rotatable is a rotating sleeve 1 with an external thread 1a with the same pitch as the internal thread 2a of the housing 2, which engages in the internal thread 2a. The rotating sleeve 1 has an internal thread 1b with a pitch of 34° for example, which is smaller than the pitch of the external thread 1a and the internal thread 2a of the housing. The difference between the thread pitches may vary. For example, in some embodiments it may range from approximately 5-30 degrees and in some embodiments it may range from approximately 10 to 15 degrees.

Mounted inside the rotating sleeve 1 is a forward-feed element 3, secured to pre-vent it from rotating relative to the housing 2. The forward-feed element has an external thread 3a which engages in the internal thread 1b of the rotating sleeve 1. Mounted on the distal or front end of the forward-feed element 3, illustrated on the left in FIG. 1A, are two lugs or cams 3f lying opposite one another attached to elastic arms 3e, which are able to engage in teeth provided on the external face of a toothed rack 5, which is able to slide axially inside the forward-feed element 3. The lugs or cams 3f of the forward-feed element 3 as well as the lugs or cams 2f connected to the housing 2 and illustrated in FIG. 3 can be moved in the proximal direction relative to the toothed rack, and the lugs 2f, 3f are pressed outwards against the elastic or spring force of the elastic arms 2e and 3e so that they can be moved away from and across one or more teeth of the toothed rack 5. However, if the lugs 2f and 3f are pushed by the biasing action of the elastic arms 2e and 3e acting in the direction towards the toothed rack 5 and thus engage with the teeth, a movement of the lugs 2f and 3f in the distal direction of the toothed rack 5 is not possible and is prevented due to the lugs 2f and 3f engaging in the teeth of the toothed rack 5.

At the proximal end, the rotating sleeve 1 is connected to a control knob 4 and is mounted in it so as to be rotatable via a circumferentially extending groove 4a provided on the internal face of the control knob 4, in which a circumferentially extending ring 1c provided on the external face of the rotating sleeve 1 engages. When the control knob 4 is pulled out of the housing 2 by a user, it drives with it the rotating sleeve 1, which is rotated relative to the housing 2 during the pulling out operation due to the external thread 1a engaging in the internal thread 2a of the housing 2. This rotation of the rotating sleeve 1 is converted into an axial movement of the forward-feed element 3 due to the engagement with the internal thread 1b of the rotating sleeve 1 by the external thread 3a of the forward-feed element 3, which is mounted so that it is prevented from rotating relative to the housing 2. Since the external thread 1a of the rotating sleeve has a bigger pitch than the internal thread 1b of the rotating sleeve, an extraction distance D, indicated in FIG. 1B, is converted into a shorter extraction distance d of the forward-feed element 3, so that small dose quantities can be precisely set.

Disposed on the external face of the forward-feed element 3 are two axially spaced stops 3c and 3d projecting radially outwardly, between which the stop element 2b connected to the housing 2 engages. In an initial position illustrated in FIG. 1A, the distal axial stop 3c of the forward-feed element lies on the distal side of the stop element 2b. The proximal stop 3d of the forward-feed element is at a distance d from the distal side of the stop element 2b.

FIG. 1B illustrates the dose setting device in accordance with the present invention after the control knob 4 has been pulled out by a distance D of 5 mm, for example, which leads to the axial movement reduced by the distance d of 0.8119 mm of the forward-feed element 3, until the stop 3d of the forward-feed element 3 lies on the distal side of the stop element 2b, as a result of which the extraction movement of the control knob 4 is restricted. The rotating sleeve 1 was rotated by −90° during this operation, for example.

As the control knob 4 is being pulled out and the forward-feed element 3 is moved in the proximal direction, the lugs or cams 3f connected to the forward-feed element 3 are moved in the proximal direction along the toothed rack 5, which is held by the lugs 2f connected to the housing so that the lugs 3f lying opposite one another are pushed backwards across one, two or more teeth of the toothed rack 5.

When a user depresses the control knob 4 and pushes it back into the housing 2, as illustrated in FIG. 1C, the retraction movement of the control knob is transmitted to the rotating sleeve 1, which, because of the threaded engagement with the housing 2, rotates relative to it by +90°, for example, and pushes it axially forwardly due to the threaded engagement with the forward-feed element 3. Movement occurs until the proximal stop 3c lies against the stop element 2b again and the front end of the rotating sleeve 1 lies against a stop 3b of the forward-feed element 3. As this takes place, the toothed rack 5 is held by the lugs 3f engaging in the teeth and is pushed together with the forward-feed element 3 in the distal direction, and the toothed rack is moved by the distance d relative to the lugs 2f connected to the housing 2, which, when the forward feed operation is complete, engage in the teeth of the toothed rack 5 which are now axially offset from the initial position illustrated in FIG. 1A in the proximal direction. This forward-feed movement of the toothed rack 5 is transmitted to the plunger 6 illustrated in FIG. 3, which is pushed into the ampoule 7 inserted in the injection device and thus applies pressure to the substance contained in the ampoule 7, e.g. insulin, so that the quantity of substance corresponding to the forward-feed movement d of the plunger 6 is discharged from the ampoule 7.

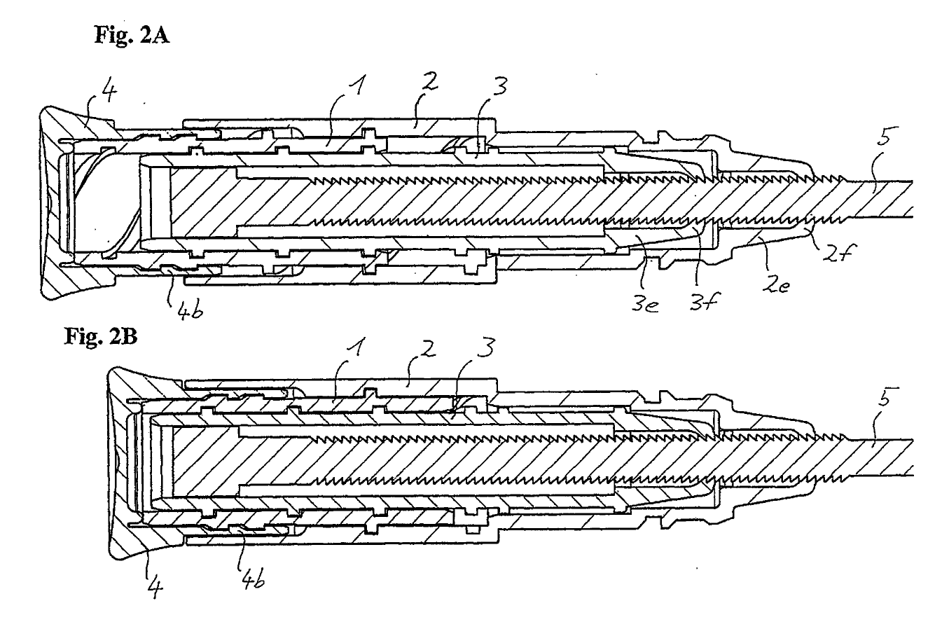

FIGS. 2A and 2B illustrate a cross-section of another embodiment of the dose setting device in accordance with the present invention in which the control knob 4 has webs 4b projecting axially into the housing 2 or a cylindrical, circumferentially extending element 4b which can be pushed into a matching recess of the housing 2. As the control knob 4 is being pulled out, therefore, the external face of the rotating sleeve 1 is not visible, as may be seen from FIG. 1B, but the external face of the element 4b is, as illustrated in FIG. 2A.

Markings may be provided on both the external face of the rotating sleeve 1 and the external face of the element 4b to display information for a user relating to a dose set by the extraction movement out of the housing 2, e.g. by colored markings, rings or other indicia.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A dose setting device for an injection device comprising a housing, a forward-feed element and a setting element, the dose setting device comprising a rotating sleeve connected to the setting element and having an external thread and an internal thread with a selected different pitch from the external thread, wherein one thread of the rotating sleeve engages with the injection device and the other thread engages with the forward-feed element whereby a dose setting movement of the setting element is different from a dose setting movement of the forward-feed element.

2. A dose setting device for an injection device, the dose setting element comprising a setting element, a generally cylindrical rotating sleeve connected to the setting element and having an external thread and an internal thread with a different pitch from the external thread, wherein one thread of the rotating sleeve engages with a thread of a part of the injection device, and the other thread engages with a thread of a forward-feed element, whereby a setting movement of the setting element results in a reduced or increased dose setting movement of the forward-feed element due to the two threaded engagements.

3. The dose setting device as claimed in claim 2, wherein the pitch of the thread of the rotating sleeve which engages with the forward-feed element is smaller than the pitch of the other thread of the rotating sleeve.

4. The dose setting device as claimed in claim 3, wherein the difference in the thread pitches ranges from 5-30 degrees.

5. The dose setting device as claimed in claim 2, wherein the pitch of the thread of the rotating sleeve which engages with the forward-feed element is bigger than the pitch of the other thread of the rotating sleeve.

6. The dose setting device as claimed in claim 5, wherein the difference in the thread pitches ranges from 5-30 degrees.

7. The dose setting device as claimed in claim 2, wherein the forward-feed element is mounted so that it can not rotate relative to the housing.

8. The dose setting device as claimed in claim 2, wherein an internal thread is provided on the housing in which the external thread of the rotating sleeve engages, and the forward-feed element has an external thread which engages in the internal thread of the rotating sleeve.

9. The dose setting device as claimed in claim 2, wherein the rotating sleeve is fixedly or rotatably connected to the setting element.

10. The dose setting device as claimed in claim 2, wherein the thread pitches of the rotating sleeve, the housing and the forward-feed element are selected so that the threads are not retained by friction.

11. The dose setting device as claimed in claim 2, further comprising a spring element which acts in or opposite the direction of rotation of the rotating sleeve.

12. The dose setting device as claimed in claim 11, wherein the spring element is a torsion spring.

13. The dose setting device as claimed in claim 2, wherein the external thread of the rotating sleeve is disposed coaxially with and at least partially overlapping the internal thread of the rotating sleeve in the radial direction.

14. The dose setting device as claimed in claim 2, further comprising at least one catch lug or catch cam on an elastic element carried by the forward-feed element.

15. The dose setting device as claimed in claim 2, further comprising a toothed rack able to move axially inside the dose setting device and mounted inside the forward-feed element, the rack comprising at least one or two rows of teeth on an external face in which at least one of at least one elastically mounted catch lug of the forward-feed element and at least one elastically mounted catch lug connected to the housing can engage.

16. The dose setting device as claimed claim 2, further comprising at least one radial or axial stop on at least one of the forward-feed element, the rotating sleeve or the housing to restrict at least one of a radial or axial movement of at least one of the forward-feed element and the rotating sleeve relative to the housing.

17. The dose setting device as claimed in claim 2, further comprising marking on at least one of an external face of the setting element and an external face of the rotating sleeve to display a set dose.

18. An injection device comprising a housing, a forward-feed element, a setting element and a dose setting device comprising a rotating sleeve connected to the setting element and having an external thread and an internal thread with a selected different pitch from the external thread, wherein one thread of the rotating sleeve engages with the injection device and the other thread engages with the forward-feed element whereby a setting movement of the setting element is different from a dose setting movement of the forward-feed element, wherein the difference in the thread pitches ranges from 5-30 degrees.

19. An injection device comprising a housing, a forward-feed element, a setting element and dose setting device, the dose setting device comprising a rotating sleeve connected to the setting element and having an external thread and an internal thread with a selected different pitch from the external thread, wherein one thread of the rotating sleeve engages with the injection device and the other thread engages with the forward-feed element whereby a dose setting movement of the setting element is different from a dose setting movement of the forward-feed element.

20. The injection device as claimed in claim 19, further comprising a toothed rack able to move axially inside the dose setting device and carried inside the forward-feed element, the rack comprising at least one or two rows of teeth on an external face in which at least one of at least one elastically mounted catch lug of the forward-feed element and at least one elastically mounted catch lug connected to the housing can engage.

21. The injection device as claimed claim 20, further comprising at least one radial or axial stop on at least one of the forward-feed element, the rotating sleeve or the housing to restrict at least one of a radial or axial movement of at least one of the forward-feed element and the rotating sleeve relative to the housing.

22. The injection device as claimed claim 21, wherein the difference in the thread pitches ranges from 5-30 degrees.
